(19) United States
(12) Patent Application Publication
(10) Pub. No.: US 2007/0156215 A1
(43) Pub. Date: Jul. 5, 2007

(54) Dilating lead tip

(76) Inventors: Marc Jensen, Los Gatos, CA (US); Todd Thompson, San Jose, CA (US)

Correspondence Address: BOZICEVIC, FIELD & FRANCIS LLP (PRTS);(PROTEUS BIOMEDICAL,INC), 1900 UNIVERSITY AVENUE, SUITE 200, EAST PALO ALTO, CA, 94303, US

(21) Appl. No.: 11/646,183

(22) Filed: Dec. 26, 2006

Related U.S. Application Data
(60) Provisional application No. 60/755,047, filed on Dec. 29, 2005.

(51) Int. Cl. A61N 1/05

(52) U.S. Cl. 607/116; 607/119; 607/122

(57) ABSTRACT

Tapered pacing lead tip shapes and fixation devices, as well as methods for making and using the same, are provided. The inventive leads include features which optimize placement and retention of the leads, as well as features which improve function of the lead. Also provided are implantable devices and systems, as well as kits containing such devices and systems or components thereof, which include the inventive dilating lead tip designs and segmented electrode structures.

### CROSS REFERENCE TO RELATED APPLICATIONS

Pursuant to 35 U.S.C. § 119 (e), this application claims priority to: U.S. Provisional Application Ser. No. 60/755,047 filed on Dec. 29, 2005, the disclosure of which priority application is herein incorporated by reference.

### INTRODUCTION

### Background

Pacemakers and other implantable medical devices find wide-spread use in today's health care system. A typical pacemaker includes stimulating electrodes that are placed in contact with heart muscle, detection electrodes placed to detect movement of the heart muscle, and control circuitry for operating the stimulating electrodes based on signals received from the detection electrodes. Thus, the pacemaker can detect abnormal (e.g., irregular) movement and deliver electrical pulses to the heart to restore normal movement.

Pacing leads implanted in vessels in the body have traditionally had electrodes at the distal tip of the lead to increase the probability that the lead electrodes would contact excitable tissue, usually within the heart's right ventricle. The most popular designs have soft flexible tines at the tip to entangle trabecular tissue in the ventricle, or screw tips that extend out from the tip of the pacing lead. Implantation of leads in the coronary veins is increasingly common as heart failure therapies are more widely used and there is an increased need for stimulating the left side of the heart, either alone or in conjunction with right sided stimulation. Previous methods for achieving left ventricular pacing required placement of an epicardial lead, via an invasive thoracotomy or thoracoscopy. More recently intravascular leads have been developed. The ability to implant leads in the coronary veins in a non-invasive way is of great utility in controlling the timing of cardiac pacing, especially in optimizing pacing therapies, e.g., such as CRT (cardiac resynchronization therapy).

The present invention is for pacing leads that are designed for implantation in the small veins of the heart.

Relevant Literature

Publications of interest include: U.S. Pat. Nos. 5,476,498; 5,531,781; 5,755,765; 5,755,766; 5,935,160; 6,136,021; 6,178,356; 6,129,750; 6,385,492; 6,556,873; 6,584,362; 6,662,055; 6,697,676; 6,714,823; 6,882,886; 6,961,621; 7,139,614; International Application No. PCT/US2002/0173785; International Publication No. WO2006/116284.

### SUMMARY

The present invention provides tapered pacing lead tip shapes and fixation devices, as well as methods for making and using the same. The leads of the present invention are designed to sense and pace the left heart from a position within the cardiac veins. The inventive leads include features which optimize placement and retention of the leads, as well as features which improve the contact between the electrodes and the heart wall. The tip design is tapered to facilitate placement in distal veins, and has structural elements on its outer surface that provide for either reduction of contact friction or reduction of fluid backpressure during implantation. These structural elements can include grooves to allow fluid pressure to be relieved while pushing the lead forward. The tapered tip can also include ridges with soft flexible teeth to help wedge the lead into the vein. The tapered tip may or may not have electrodes on the tapered section of the lead tip.

The tapered tip can be combined with a lead body that has lateral deflection of the electrodes along the body of the lead, which is designed to bring the electrodes in close proximity to the epicardial surface of the heart. Electrodes which are oriented toward and make contact with the epicardial surface of the heart increase effectiveness of the stimulation, limit power use, and avoid unwanted excitation of nerves. The lead body can also have a diameter increase from distal to proximal, with an overall diameter range from 2F to 9F. The subject device can have a tapered flexible tip with an open lumen, or can have a closed-ended tapered flexible tip at its distal end.

Due to the tortuous nature of the vessels in the body, following implantation the rotational orientation of one electrode can not be predetermined in many currently employed devices. As such, many currently employed lead devices use cylindrical electrode designs that are conductive to tissue around the entirety of the diameter of the lead. This insures that some portion of the cylindrical electrode contacts excitable tissue when implanted. Despite the multiple devices in which cylindrical continuous ring electrodes are employed, there are disadvantages to such structures, including but not limited to: undesirable excitation of non-target tissue, e.g., which can cause unwanted side effects, increased power use, etc.

An innovative way to address this problem is to employ segmented electrode structure, in which the circular band electrode is replaced by an electrode structure made up of two or more individually activatible and electrically isolated electrode structures that are configured in a discontinuous band. The subject devices can have one or more electrodes that form solid cylinders connected through the length of the lead. The device can also have a multiplicity of segmented electrode structures made up of an integrated circuit conductively coupled to two or more electrodes, where each electrode can be individually activated. Such segmented electrode structures are disclosed in published PCT application Publication Nos. WO 2006/069322 and WO2006/029090; the disclosures of which are herein incorporated by reference.

Also provided are implantable devices and systems, as well as kits containing such devices and systems or components thereof, which include the inventive lead designs and segmented electrode structures.

### BRIEF DESCRIPTION OF THE FIGURES

FIG. 1 shows a tapered tip on a pacing lead.

FIG. 2A shows a tapered tip on a pacing lead with two ridges on each side of the tip. The ridges have teeth on the edges.

FIG. 2B shows the detail of the teeth on the ridges. Also visible is an open lumen.

FIG. 3 shows a tapered tip on a pacing lead with a groove along the surface of the tip.

FIG. 4A shows a tapered tip on a pacing lead with a groove forming a spiral along the surface of the tip.

FIG. 4B shows a tapered tip with a spiral groove on a pacing lead with proximal section larger than the following body section.

FIG. 4C shows a tapered tip on a pacing lead in cross-sectional view.

FIG. 5 shows a tapered tip on a pacing lead with proximal section with lateral deflections between the body sections containing electrodes.

FIG. 6 shows a tapered tip on a pacing lead with proximal section increasing in diameter in the more proximal sections.

FIG. 7 shows a flexible electrode pattern.

FIG. 8 shows a segmented electrode section.

FIG. 9 shows a pacing lead implanted in a heart.

### DETAILED DESCRIPTION

In describing the invention, aspects of elongated implantable flexible structures, or lead tip shapes and fixation devices, as well as methods for making and using the same are reviewed first in greater detail, both generally and in terms of figures of certain embodiments of the invention. Next, embodiments of devices and systems, including the use of implantable addressable segmented electrodes and implantable pulse generators, are described, as well as methods of using such devices and systems in different applications. Also provided is a description of kits that incorporate aspects of the invention.

Dilating Lead Tips

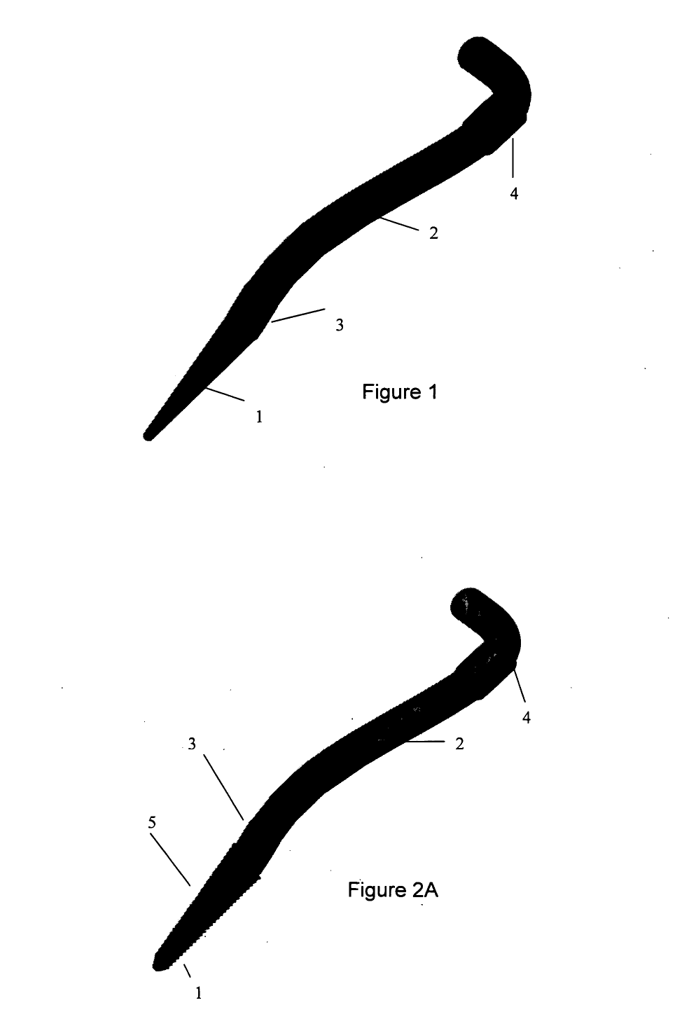

As summarized above, FIG. 1 provides a representation of the invention, an elongated implantable flexible structure, e.g. a pacing cardiovascular lead, with a flexible tapered tip 1, a multiplicity of electrodes 3, 4 and a lead body 2. The taper angle of the tip (relative to the normal of the axis of the lead) can vary, and in certain embodiments can range from about 74° to about 88°. The taper may be constant or the taper may also be curved. The advantage of the taper is to provide distal flexibility, with increased bending and compression modulus proximally. Increased compression strength allows the device to push through valves in the vascular system, and allows the device to dilate small vessels.

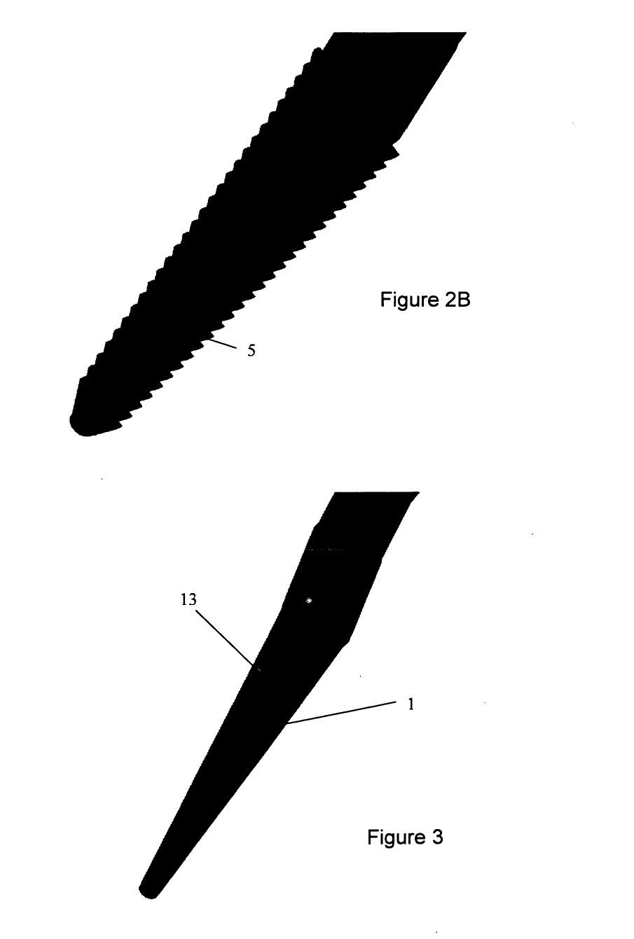

FIG. 2A provides a representation of an elongated implantable flexible structure, e.g. a pacing lead, with a tapered tip 1, a multiplicity of electrodes 3, 4 and a lead body 2 with one or more ridges 5 on the outside of the taper. The ridges reduce the contact friction of the device as it is inserted into the vasculature. The ridges also allow some fluid to pass along the lead body to reduce the fluid backpressure.

FIG. 2B provides a representation of the “teeth” feature on the edges of the ridges 5 to help grip the inner diameter of the vasculature.

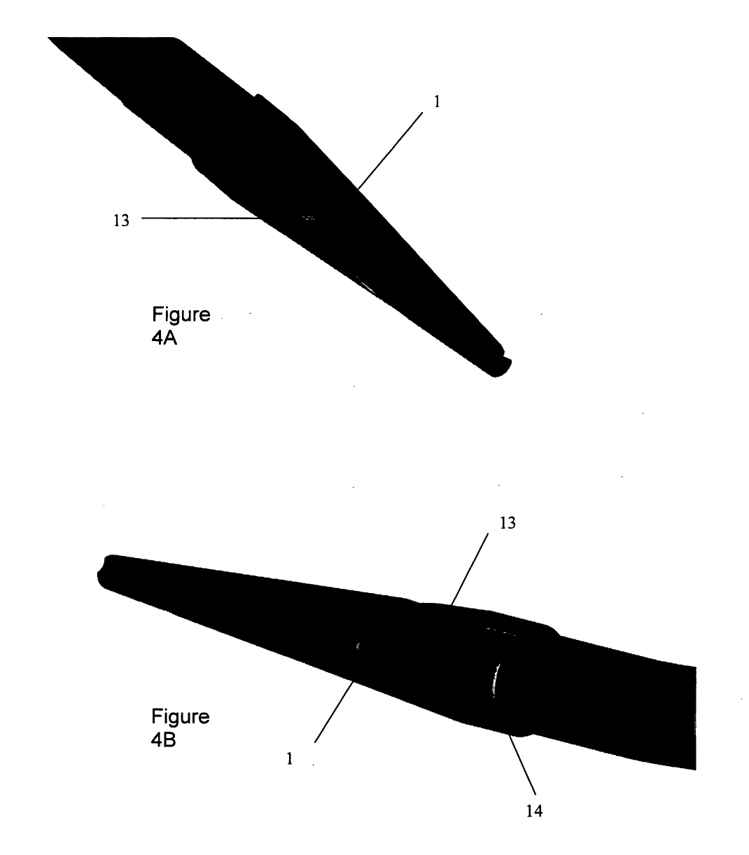

FIG. 3 provides a representation of the tapered tip 1 with one or more grooves 13 on the outside of the taper. The grooves also allow some fluid to pass along the lead body to reduce the fluid backpressure, which is necessary because the direction of blood flow in the coronary veins is opposite to the direction of lead implantation, and this feature counteracts the tendency of the blood flow to dislodge the lead.

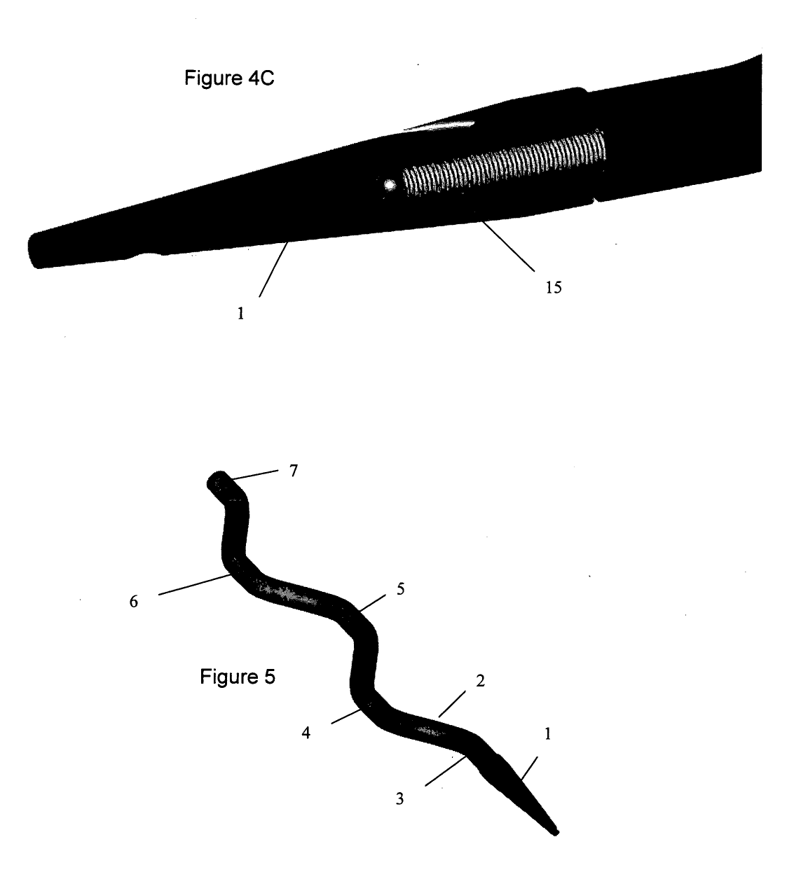

FIG. 4A provides a representation of one or more grooves 13 in a spiral pattern on the outside of the tapered tip 1. The spiral keeps the grooves from being plugged by the elastic vessels that the lead is placed in during placement of the lead, FIG. 4B provides a representation of a larger proximal section 14 of the tapered tip 1. The larger section and more specifically the proximal edge of the structure provide a feature to lock the position of the lead as the vessels recoil after dilation from the lead tip's passage. A spiral groove 13 allows fluid (blood) to pass by the lead, reducing the force that would expel the lead. FIG. 4C provides a representation of the tapered flexible tip 1 with a closed-ended solid section at its distal end. An inner structure 15 provides a closed-ended lumen for a stylet to transmit additional pushing forces to the tapered flexible tip. The structure 15 can be a coiled metallic coil or a polymer core.

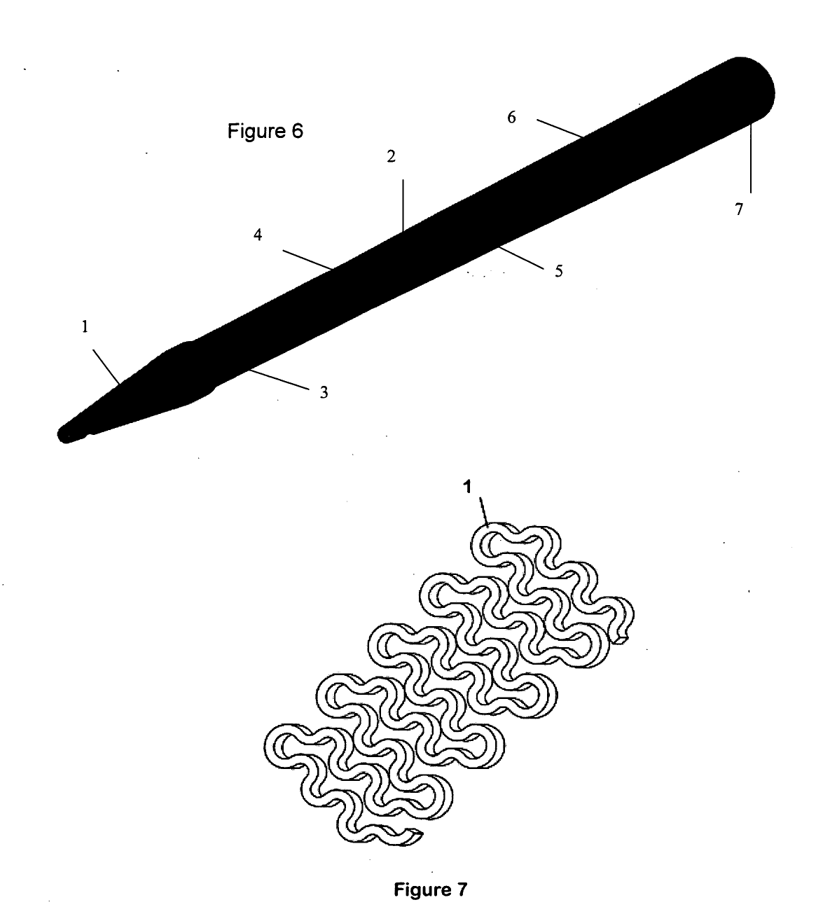

FIG. 5 and FIG. 6 show features of the inventive elongated implantable flexible structures, e.g. leads, which improve contact between electrodes on the lead and the wall of the coronary veins. FIG. 5 provides a representation of the tapered tip 1 with a lateral deflection of the lead body 2 between the multiplicities of electrodes 3, 4, 5, 6, 7. The lateral deflection provides better contact between the lead body and the vessel wall in the more proximal vessel sections. FIG. 6 provides a representation of the tapered tip 1 with an increasing diameter lead body 2 between the multiplicities of electrodes 3, 4, 5, 6, 7. The increasing diameter also provides better contact between the lead body in the more proximal vessel sections. In addition, the increasing diameter of the lead body provides distal flexibility with increased bending and compression modulus proximally. Increased compression strength allows the device to push through valves in the vascular system to assist in placement of the lead, and allows the device to dilate small vessels. FIG. 4C and FIG. 6 provide a representation of the closed-ended tapered flexible tip 1, which has a closed-ended lumen at its distal end. FIG. 2B and FIG. 3 provide a representation of the tapered tip 1 with an open lumen.

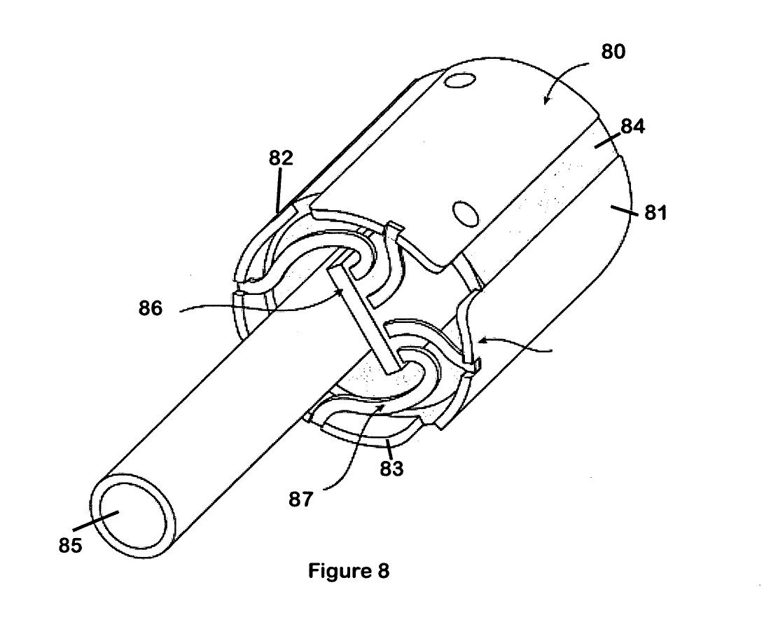

Embodiments of the invention further include the use of electrode configurations as described below. FIG. 7 provides a representation of an electrode configuration with a flexible structure that is a complete cylinder of surface area 8 mm². Solid cylinder electrodes may range from about 2 mm² to about 15 mm², such as from about 5 mm² to about 10 mm² may be employed. Solid cylinder electrodes may be connected with a conductor through the length of the lead body. Additional electrode configurations of interest include, but are not limited to: curved electrodes, bent electrodes, segmented electrodes, helical electrodes, and the like.

Segmented electrodes can have a small surface area and still adequately excite the tissue that needs to be excited. For example, electrodes having surface areas ranging from about 0.1 mm² to about 4.0 mm², such as from about 0.5 mm² to about 3.0 mm² may be employed. Despite their small surface area, excitation of that tissue that needs to be excited is achieved. Because of the segmented nature of the structures, excitable tissue will be contacted regardless of how the device is placed in the vessel. The quadrant arrangement of electrodes allows the administration of pacing current via electrodes oriented at a preferred direction, for example, away from nerves, or facing an electrode configured to sink the pacing current. Such precise pacing allows low-power pacing and minimal tissue damage caused by the pacing signal. With the reduced surface area of the segmented electrode, the impedance increases thereby reducing the current drain on the pacemaker, which can lead to improved longevity of the device.

The inventive use of separately addressable quadrants on a multiple electrode leads allows a number of other clinical advantages. In many cases, the present invention allows patients who would be unresponsive using prior art devices to become responsive to treatment. For example, multiple potential resynchronization positions along the lead allows for selection in real time of the most advantageous pacing, without requiring repositioning of the lead. Synergistic use of multiple points of stimulation are also available, again without any further lead positioning. Currently available techniques require difficult and often unsuccessful repositioning of the lead when an effective resynchronization positioning is not achieved. Because of difficulties in variations of anatomical features, and limitations in time available for repositioning, often results are suboptimal or poor. Additional advantages include the ability to achieve fine measurement of conduction velocity in different axes.

Embodiments of the segmented electrode structures may include one or more of the above features, or others. In further describing the invention, embodiments of the structures are now reviewed in greater detail in terms of the figures.

As mentioned above, FIG. 8 provides a representation of a segmented electrode satellite structure according to an embodiment of the invention. The size of the cardiac pacing electrodes of the present invention may vary, and in certain embodiments range from about 0.1 mm² to about 4 mm² in area, e.g., about 1.5 mm² in area. The electrodes (80, 81, 82 and 83) can be positioned relative to the integrated circuit (IC) (86) in a variety of different formats, e.g., circumferentially around the IC and/or the body of a lead, or they could be distributed longitudinally along the length of the lead body, extending from the connection from the IC or they could be arranged in a pattern that improves tissue contact.

By segmented electrode structure is meant an electrode structure that includes two or more, e.g., three or more, including four or more, disparate electrode elements. Embodiments of segmented electrode structures are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006; 60/807,289 titled “High Phrenic, Low Capture Threshold Pacing Devices and Methods,” filed Jul. 13, 2006, and U.S. Patent Application 60/865,760 titled “Housing for Implantable Segmented Electrode” filed on Nov. 14, 2006; the disclosures of the various segmented electrode structures of these applications being herein incorporated by reference. In these embodiments, the support may include a recess for each electrode element of the segmented electrode structure. As such, the support may include 2 or more, 3 or more, 4 or more, etc., where each recess is configured to receive an electrode element (i.e., an electrode inset).

In certain embodiments, the electrodes are “addressable” electrode structures. Addressable electrode structures include structures having one or more electrode elements directly coupled to control circuitry, e.g., present on an integrated circuit (IC). Addressable electrode structures include satellite structures that include one more electrode elements directly coupled to an IC and configured to be placed along a lead. Examples of addressable electrode structures that include an IC are disclosed in application Ser. Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various addressable electrode structures of these applications being herein incorporated by reference. In these embodiments where the supports are configured to support an addressable electrode structure that includes an IC, the support may include IC holding elements that immobilize an IC inside the support. IC holding elements of interest include, but are not limited clamps, clips, notches configured to receive a portion (e.g., edge) of an IC, etc.

The materials of construction of the conductive members, e.g., electrodes, for use with the presently described ICs may be primarily platinum, or platinum alloy, including platinum, 5% iridium, platinum 10% iridium, or platinum, 20% iridium. Additional appropriate platinum alloys include, but are not limited to: platinum 8% tungsten, platinum nickel, and platinum rhodium. An additional material for the electrode of the present invention can be titanium. The titanium can be plated with platinum or the platinum alloys previously described. Corrosion resistant alloys can also be deposited by RF Sputtering, E beam vapor deposition or chemical vapor deposition, among other methods. In addition to titanium, base electrode materials can include Stainless Steel 316SS, cobalt based supper alloys MP35N or Tantalum. The electrode can also be electroformed.

The electrodes may be fabricated from bulk cold worked alloys. In addition, the electrodes can be formed wholly from thin film deposition processes. The electrodes formed from bulk metal or alloys can take advantage of a fine microstructure formed by cold working to the final thickness. Refined microstructures typically increase the yield point of the material and the fatigue life of the material.

Electrodes formed by thin film processes can be made with the same class of materials described previously. The electrodes can also be fabricated as layered structures that exploit different material characteristics for optimum performance. High strength metals or alloys can be deposited for optimum strength as base layer. Additional corrosion resistant layers could be formed above the base layers. The final coatings could be materials that enhance the electrodes ability to transfer charge to tissue or to sense electrical signals. In addition other coatings on the electrode could enable chemical sensing or pH measurements.

The fabrication process from bulk metals or alloys can be done by any convenient method, such as methods employed for fabrication of cardiovascular stents and other passive mechanical devices. The electrode can be manufactured by laser cutting, Electric Discharge Machining (EDM), photochemical etching, or by stamping and forming or a combinations of those fabrication processes. In addition, that electrode can be chemical etched, or electropolished to produce a smooth surface. Smooth surfaces are desirable for fatigue resistant devices to reduce the number of potential crack initiation sites.

Additionally the electrode can be formed by sputtering a suitable metal or alloy on fabric that would cover the outside surface of the medical device. The sputtered area can then be plated to additional thickness if required. This allows the fabric to perform two functions-first to reinforce the lead from applied mechanical effects and second to provide a flexible substrate for a conductive electrode. This configuration reduces the abrupt change in bending stiffness that results from a change in materials along the length. This conductive area is then connected to the IC chip with a flexible conductive member.

In the present invention, the surface of the conductive member(s), e.g., electrodes, may be different than the bulk material. The surface that is exposed to the blood stream should survive corrosion and electrolytic corrosion that occurs in that environment. In addition the surface should maximize the charge transfer to the tissue for pacing. The surface, in certain embodiment, will optimize sensing of electrical signals. The surface can also provide the ability to sense chemical species or pH changes.

The surface coating may include elements of in the noble metal family including the alloys, oxides and nitrides (platinum, platinum iridium, titanium nitride, and iridium oxide). In addition these materials can increase the micro roughness of the electrode, increasing the microscopic surface area. This improves the capacitive charge transfer ability of the electrode.

In FIG. 8, quadrant electrodes 80, 81, 82 and 83 are joined together with PEEK material 84. Guide wire lumen 85, which may accommodate a bus which is coupled to a number (e.g., eight) of electrode satellites, runs beneath I.C. 86, which is connected to the electrodes with the flexible members 87. The electrode satellites can be segmented electrodes. The main conductors are not shown in this figure.

Vascular Leads

Embodiments of the invention also include medical carriers that include one or more electrode satellite structures, e.g., as described above. Carriers of interest include, but are not limited to, vascular lead structures, where such structures are generally dimensioned to be implantable and are fabricated from a physiologically compatible material. In some embodiments of the invention, the medical carriers can be employed with a neurological, muscular, gastrointestinal, skeletal, or pulmonary medical device. With respect to vascular leads, a variety of different vascular lead configurations may be employed, where the vascular lead in certain embodiments is an elongated tubular, e.g., cylindrical, structure having a proximal and distal end. The proximal end may include a connector element, e.g., an IS-1 connector, for connecting to a control unit, e.g., present in a “can” or analogous device. The lead may include one or more lumens, e.g., for use with a guidewire, for housing one or more conductive elements, e.g., bus, wires, etc. The distal end may include a variety of different features as desired, e.g., a securing means, etc.

In certain embodiments of the subject systems, one or more sets of electrode satellites as described above are electrically coupled to at least one elongated conductive member, e.g., an elongated conductive member present in a lead, such as a cardiovascular lead. In certain embodiments, the elongated conductive member is part of a multiplex lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex leads include 3 or fewer wires, such as only 2 wires or only 1 wire. Multiplex lead structures of interest include those described in Application Serial Nos.: 10/734,490 titled “Method and System for Monitoring and Treating Hemodynamic Parameters” filed on Dec. 11, 2003; PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various multiplex lead structures of these applications being herein incorporated by reference. In some embodiments of the invention, the devices and systems may include onboard logic circuitry or a processor, e.g., present in a central control unit, such as a pacemaker can. In these embodiments, the central control unit may be electrically coupled to the lead by a connector, such as a proximal end IS-1 connection.

The leads may further include a variety of different effector elements, which elements may employ the satellites or structures distinct from the satellites. The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US20051046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/USUS05/39535 titled “Cardiac Motion Characterization by Strain Gauge”. These applications are incorporated in their entirety by reference herein.

Implantable Pulse Generators

Embodiments of the invention further include using implantable pulse generators. Implantable pulse generators may include: a housing which includes a power source and an electrical stimulus control element; one or more vascular leads as described above, e.g., 2 or more vascular leads, where each lead is coupled to the control element in the housing via a suitable connector, e.g., an IS-1 connector. In certain embodiments, the implantable pulse generators are ones that are employed for cardiovascular applications, e.g., pacing applications, cardiac resynchronization therapy applications, etc. As such, in certain embodiments the control element is configured to operate the pulse generator in a manner so that it operates as a pacemaker, e.g., by having an appropriate control algorithm recorded onto a computer readable medium of a processor of the control element. In certain embodiments the control element is configured to operate the pulse generator in a manner so that it operates as a cardiac resynchronization therapy device, e.g., by having an appropriate control algorithm recorded onto a computer readable medium of a processor of the control element.

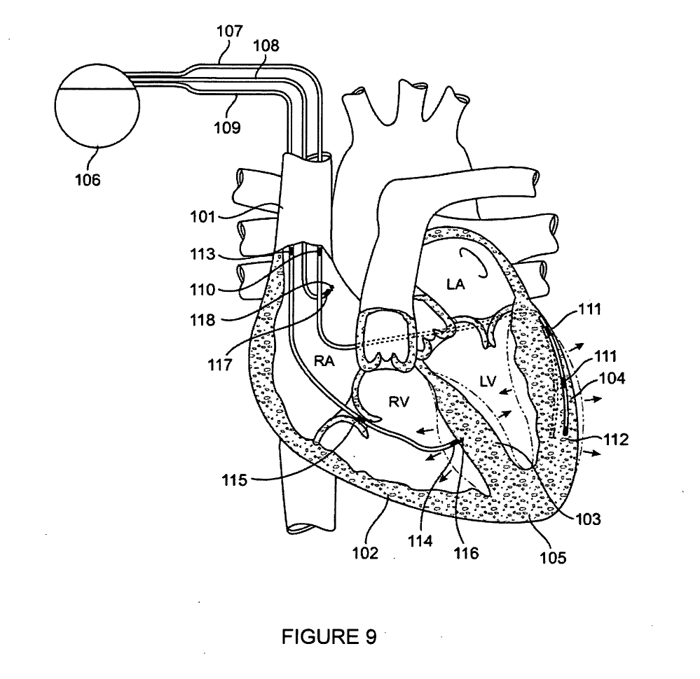

A representative system in which an implantable pulse generator and hermetically sealed integrated structures find use according to an embodiment of the invention is depicted in FIG. 9, which provides a cross-sectional view of the heart with of an embodiment of a cardiac resynchronization therapy (CRT) system. The system includes a pacemaker can 106 that includes a control element (e.g., processor) and a power source, a right ventricle electrode lead 109, a right atrium electrode lead 108, and a left ventricle cardiac vein lead 107. Also shown are the right ventricle lateral wall 102, interventricular septal wall 103, apex of the heart 105, and a cardiac vein on the left ventricle lateral wall 104.

The left ventricle electrode lead 107 is comprised of a lead body and one or more electrodes 110, 111, and 112. Each of the electrodes includes a hermetically sealed integrated circuit. Having multiple distal electrodes allows a choice of optimal electrode location for CRT. In a representative embodiment, electrode lead 107 is constructed with the standard materials for a cardiac lead such as silicone or polyurethane for the lead body, and MP35N for the coiled or stranded conductors connected to Pt—Ir (90% platinum, 10% iridium) electrodes 110, 111 and 112. Alternatively, these device components can be connected by a multiplex system (e.g., as described in published United States Patent Application publication nos.: 20040254483 titled “Methods and systems for measuring cardiac parameters”; 20040220637 titled “Method and apparatus for enhancing cardiac pacing”; 20040215049 titled “Method and system for remote hemodynamic monitoring”; and 20040193021 titled “Method and system for monitoring and treating hemodynamic parameters; the disclosures of which are herein incorporated by reference), to the proximal end of electrode lead 107. The proximal end of electrode lead 107 connects to a pacemaker 106.

The electrode lead 107 is placed in the heart using standard cardiac lead placement devices which include introducers, guide catheters, guidewires, and/or stylets. Briefly, an introducer is placed into a cephalic or subclavian vein. A guide catheter is placed through the introducer and used to locate the opening to the coronary sinus in the right atrium. A guidewire is then used to locate a left ventricle cardiac vein. The electrode lead 107 is slid over the guidewire into the left ventricle cardiac vein 104 and tested until an optimal location for CRT is found. Once implanted a multi-electrode lead 107 still allows for continuous readjustments of the optimal electrode location.

The electrode lead 109 is placed in the right ventricle of the heart with an active fixation helix at the end 116 which is embedded into the cardiac septum. In this view, the electrode lead 109 is provided with one or multiple electrodes 113, 114, 115.

Electrode lead 109 is placed in the heart in a procedure similar to the typical placement procedures for cardiac right ventricle leads. Electrode lead 109 is placed in the heart using the standard cardiac lead devices which include introducers, guide catheters, guidewires, and/or stylets. Electrode lead 109 is inserted into a cephalic or subclavian vein, through the superior vena cava, through the right atrium and down into the right ventricle. Electrode lead 109 is positioned under fluoroscopy into the location the clinician has determined is clinically optimal and logistically practical for fixating the electrode lead 109. Under fluoroscopy, the active fixation helix 116 is advanced and screwed into the cardiac tissue to secure electrode lead 109 onto the septum. The electrode lead 108 is placed in the right atrium using an active fixation helix 118. The distal tip electrode 118 is used to both provide pacing and motion sensing of the right atrium.

Summarizing aspects of the above description, in using the implantable pulse generators of the invention, such methods include implanting an implantable pulse generator e.g., as described above, into a subject; and the implanted pulse generator, e.g., to pace the heart of the subject, to perform cardiac resynchronization therapy in the subject, etc. The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

During operation, use of the implantable pulse generator may include activating at least one of the electrodes of the pulse generator to deliver electrical energy to the subject, where the activation may be selective, such as where the method includes first determining which of the electrodes of the pulse generator to activate and then activating the electrode. Methods of using an IPG, e.g., for pacing and CRT, are disclosed in Application Serial Nos.: PCT/US2005/031559 titled “Methods and Apparatus for Tissue Activation and Monitoring,” filed on Sep. 1, 2006; PCT/US2005/46811 titled “Implantable Addressable Segmented Electrodes” filed on Dec. 22, 2005; PCT/US2005/46815 titled “Implantable Hermetically Sealed Structures” filed on Dec. 22, 2005; 60/793,295 titled “High Phrenic, Low Pacing Capture Threshold Implantable Addressable Segmented Electrodes” filed on Apr. 18, 2006 and 60/807,289 titled “High Phrenic, Low Capture Threshold Pacing Devices and Methods,” filed Jul. 13, 2006; the disclosures of the various methods of operation of these applications being herein incorporated by reference and applicable for use of the present, devices.

Devices and Systems

Aspects of the invention include devices and systems, including implantable medical devices and systems. The devices and systems may perform a number of different functions, including but not limited to electrical stimulation applications, e.g., for medical purposes. The systems of the invention may be viewed as systems for communicating information within the body of subject, e.g., human, where the systems include both a first implantable medical device, such as an IPG device described above, that includes a transceiver configured to transmit and/or receive a signal; and a second device comprising a transceiver configured to transmit and/or receive a signal. The second device may be a device that is inside the body, such as an implantable medical device, or may be on a surface of the body or separate from the body during use. In some embodiments the device may be a neurological device, a muscular device, a gastrointestinal device, a skeletal device, or a pulmonary device.

Also provided are methods of using the systems of the invention. The methods of the invention generally include: providing a system of the invention, e.g., as described above, that includes first and second medical devices, one of which may be implantable; and transmitting a signal between the first and second devices. In certain embodiments, the transmitting step includes sending a signal from the first to said second device. In certain embodiments, the transmitting step includes sending a signal from the second device to said first device. The signal may transmitted in any convenient frequency, where in certain embodiments the frequency ranges from about 40° to about 405 MHz. The nature of the signal may vary greatly, and may include one or more data obtained from the patient, data obtained from the implanted device on device function, control information for the implanted device, power, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Kits

Also provided are kits that include the subject dilating lead tips for implantation, and segmented electrode structures, as part of one or more components of an implantable device or system, such as the devices and systems reviewed above. In certain embodiments, the kits further include at least a control unit, e.g., in the form of a pacemaker can. In certain of these embodiments, the structure and control unit may be electrically coupled by an elongated conductive member. In certain embodiments, the segmented electrode sealed structure may be present in a lead, such as a cardiovascular lead.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described above, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

1. An elongated implantable flexible structure comprising a proximal and distal end, where said structure comprises a closed-ended tapered flexible tip at its distal end.

2. The elongated implantable flexible structure according to claim 1, wherein said tapered flexible tip comprises a structural element on its outer surface that provides for at least one of:
reduction of contact friction during implantation of said structure; and
reduction of fluid backpressure during implantation of said structure.

3. The elongated implantable flexible structure according to claim 2, wherein said structural element comprises one or more ridges.

4. The elongated implantable flexible structure according to claim 3, wherein said one or more ridges further comprise teeth.

5. The elongated implantable flexible structure according to claim 1, wherein said structural element comprises a groove.

6. The elongated implantable flexible structure according to claim 5, wherein said grooves is a spiral groove.

7. The elongated implantable flexible structure according to claim 1, wherein said tapered flexible tip is curved.

8. The elongated implantable flexible structure according to claim 1, wherein said tapered flexible tip has a taper angle between about 74° and about 88°.

9. The elongated implantable flexible structure according to claim 1, wherein said structure comprises at least one electrode.

10. The elongated implantable flexible structure according to claim 9, wherein said structure comprises two or more electrode satellites.

11. The elongated implantable flexible structure according to claim 10, wherein said satellites are coupled to a bus within said structure.

12. The elongated implantable flexible structure according to claim 11, wherein said electrode satellites are segmented electrodes.

13. The elongated implantable flexible structure according to claim 10, wherein said structure comprises one or more lateral deflections of said electrode satellites.

14. The elongated implantable flexible structure according to claim 1, wherein said structure is a cardiovascular lead.

15. An elongated implantable flexible structure have a proximal end and a distal end, where said structure comprises a tapered flexible tip at its distal end that comprises a structural element on its outer surface that provides for at least one of:
reduction of contact friction during implantation of said structure; and
reduction of fluid backpressure during implantation of said structure.

16.-27. (canceled)

28. An implantable pulse generator comprising:
(a) a housing comprising a power source and an electrical stimulus control element; and
(b) a vascular lead according to claim 1.

29.-31. (canceled)

32. A system comprising:
(a) a first implantable pulse generator according to claim 28; and
(b) a second device configured to communicate with said implantable pulse generator.

33. (canceled)

34. A method comprising:
implanting an implantable pulse generator according to claim 28 into a subject; and
using said implanted pulse generator.

35.-37. (canceled)
